(19) United States
(12) Patent Application Publication
(10) Pub. No.: US 2019/0061772 A1
(43) Pub. Date: Feb. 28, 2019

(54) SYSTEM AND METHOD FOR MONITORING THE STATE OF HEALTH AND/OR THE WELL-BEING OF A VEHICLE OCCUPANT

(71) Applicant: Audi AG, Ingolstadt (DE)

(72) Inventor: Thomas PRINZ, Karlskron (DE)

(21) Appl. No.: 15/523,847

(22) PCT Filed: Oct. 30, 2015
(86) PCT No.: PCT/EP2015/002186

§ 371 Date: Sep. 18, 2018

(30) Foreign Application Priority Data
Nov. 3, 2014 (DE) 10 2014 115 976.4
Apr. 13, 2015 (DE) 10 2015 105 581.3

(51) Int. Cl. B60W 50/00; B60W 40/08; B60W 50/16; G05D 1/00; A61B 5/18; A61B 5/00; A61B 5/0205

(52) U.S. Cl. CPC B60W 50/0098; B60W 2050/143; B60W 50/16; G05D 1/0088; A61B 5/18; A61B 5/6804; A61B 5/681; A61B 5/7455; A61B 5/486; A61B 5/002; A61B 5/747; A61B 5/02055; A61B 5/746; B60W 2040/0872; B60W 2540/26; B60W 2040/0818; B60W 2750/308; B60W 2720/10; B60W 2540/04; B60W 2710/30; B60W 2420/42; B60W 2050/0075; B60W 40/08

(57) ABSTRACT

The present invention relates to a system and a method for monitoring the state of health of a vehicle occupant. The system comprises a control unit, which comprises the following: a receiver for the wireless reception of physiological parameters of at least one unit which is worn on the body, which comprises one or more sensors for determining one or more physiological parameters of the vehicle occupant, and a diagnostic module which is designed to derive information regarding the state of health, the state of well-being, or of illnesses, at least partially on the basis of the physiological parameters received. The control unit is also designed to provide the vehicle occupants with information regarding the state of health by means of at least one output unit of the vehicle and to initiate at least one of the following steps: adapt vehicle functions to the state, or, by means of at least one output unit of the vehicle, to suggest or interactively to carry out measures that improve the state.

### SCOPE OF THE INVENTION

The present invention relates to vehicle systems and the use thereof. In particular, it relates to a system and a method for monitoring the state of health and/or of well-being of a vehicle occupant.

### BACKGROUND AND PRIOR ART USED

In the automotive industry, the use of everything from driver assistance systems to piloted driving is steadily increasing in order to improve the comfort and safety for the driver and passengers. All these systems currently focus on the vehicle and/or the vehicle environment. The driver as a decisive factor is currently hardly ever considered. There are a few systems, such as PERCLOS (Percent Eye Closure), which monitor the driver for signs of fatigue via a camera. But these are not very reliable, because many people can also nod off momentarily, and dangerously, with their eyes open, and, therefore, these systems are currently hardly used. Also, the current prior art does not consider the potential that time spent in an automobile offers for measures in everything from health prevention and stress reduction to telemedical applications.

### SUMMARY OF THE INVENTION

The present invention is based on the object to provide an improved system and method for monitoring the state of health of a vehicle occupant, which make the time spent in the vehicle more efficient for prevention and stress reduction.

This object is achieved by means of a system according to claim 1 and a method according to claim 15. Advantageous further embodiments are indicated in the dependent claims.

The system according to the invention comprises a control unit assigned to the vehicle, particularly a vehicle-mounted control unit with the following: a receiver for the wireless reception of physiological parameters from at least one unit, which is worn on the body, which comprises one or more sensors for determining one or more physiological parameters of the vehicle occupant, including at least one physiological parameter, which represents the heartbeat, the heart rate, or the heart rate variability of the vehicle occupant. Furthermore, the control unit comprises a diagnostic module, which is set up, at least partially based on the physiological parameters received, to derive information regarding the state of health, the state of well-being, or illnesses. The vehicle may be a passenger car, but this invention is not limited to such vehicles. Instead, such a vehicle may be a truck, a train, an aircraft, or a motorcycle, for example. The occupant may be the driver or pilot of the vehicle, but may additionally or alternatively also be one or more passengers.

According to the invention, the control unit is also designed to provide the vehicle occupants, by means of at least one output unit of the vehicle, with information regarding the state of health, the state of well-being, or illness, and to initiate at least one of the following steps: To adapt vehicle functions, preferably after consulting with the occupant, to the state of health or illness, or, by means of at least one output unit of the vehicle, to suggest or interactively to carry out measures that improve the state.

Although the system according to the invention comprises a control unit assigned to the vehicle, typically a vehicle-mounted control unit, the system of the invention is based, at least partially, on the processing of physiological parameters, which are determined with the assistance of a unit to be worn on the body, for example with the assistance of a wristband equipped with the corresponding sensors. Further examples of corresponding units to be worn on the body are described below.

Among the physiological parameters that the control unit receives from the device worn on the body, there is at least one physiological parameter that represents the heartbeat, the heart rate, or the heart rate variability of the vehicle occupant. To monitor the state of health and/or well-being of a vehicle occupant, the heart rate variability measurement is an especially meaningful parameter. Heart rate variability (HRV) describes the ability of the heart to continuously change the time interval from one heartbeat to the next and thus flexibly adapt to constantly changing demands. Thus, it is a measure of the general adaptability of an organism to internal and external stimuli.

From the perspective of an automotive manufacturer, the reliance on physiological parameters that are not determined with vehicle-mounted sensors but using sensors in a unit worn on the body is unusual and not obvious, since an automotive manufacturer itself will always be eager to provide all the sensors on the vehicle. For example, there have been attempts in the prior art to integrate sensors for measuring the heart rate variability into the steering wheel. Due to the constant rotation and changes of the grip on the steering wheel, a reliable HRV measurement cannot be achieved with this, however. By integrating a unit worn on the body with sensors for determining physiological parameters into the system, these parameters, and especially heart rate variability, can be measured with greater accuracy.

Another particular advantage of the integration of at least one unit worn on the body into the system is that the physiological parameters cannot be obtained solely during the times at which the vehicle occupant is in the vehicle. Instead, with a unit worn on the body, for example a wristband, physiological data can be obtained outside the vehicle, possibly even around the clock, and said data stored and subsequently transmitted to the control unit later. In this way, the diagnostic module of the control unit can have a “history” of the physiological data provided, which can be additionally taken into account in the deriving of the state of health, of the state of well-being, or illnesses from current physiological parameters. In this manner, the validity of the derivation of the current state of well-being is enhanced. In addition, the diagnostic module is able to determine past events or to determine trends in the well-being in terms of health monitoring.

Another special feature of the system of the invention is that the control unit is set up to inform vehicle occupants of their state of health. In this respect, the system goes beyond approaches which are merely intended to check the driving capacity of a driver from the perspective of the vehicle. Also, the combination with a unit worn on the body is of particular advantage for this, because this is much better-suited than vehicle-mounted sensors for real health monitoring, namely, on one hand, due to the proximity to the body, and, on the other hand, due to the already mentioned possibility of detecting physiological data outside the vehicle as well and in the deriving of information regarding the state of health.

The inventor has recognized that a vehicle, especially a passenger car, in a certain manner, represents the ideal place for monitoring and evaluating health-related physiological parameters, because most users use their car regularly and for extended periods of time, and because the environmental conditions in the vehicle are always approximately equal, so that a number of environmental factors that may affect the diagnosis can be eliminated from the outset. Another advantage is that the privacy is maintained in a vehicle, especially a passenger car, and that this is an ideal spot for the user to be informed about the state of his/her health.

In addition, the control unit may suggest or interactively perform measures to improve the state of health by means of at least one output unit of the vehicle in some embodiments, as will be shown below by means of examples. Again, this goes far beyond pure driving capacity determinations and places the user and not the vehicle in the center of the system.

Nevertheless, the diagnostic module is preferably set up, at least partially based on the received physiological parameters, to determine signs of driver incapacity, especially signs of loss of consciousness, heart attack, stroke, circulatory collapse, or epilepsy, and, in response thereto, preferably to instruct, after checking with the occupant, an autopilot device of the vehicle to perform an emergency stop, and preferably in addition to initiate an emergency call.

In addition to the inclusion of physiological parameters representing the heartbeat, the heart rate, or the heart rate variability of the vehicle occupant, the control unit is preferably set up to receive and process physiological parameters representing the electrodermal activity. The electrodermal activity manifests itself in a short-term drop in the electrical resistance of the skin, caused by an increase in sympathetic nervous system activity with emotional-affective responses. The change in the electrical conductivity of the skin is due, in this case, to increased perspiration, which is controlled by the sympathetic nervous system. Especially in combination with the heart rate variability, the electrodermal activity thus becomes a very sensitive criterion for deriving information relating to the state of health or well-being of the vehicle occupant.

Additionally or alternatively, the physiological parameters received represent a movement or acceleration of the occupant. Information regarding movement and acceleration provide valuable additional information for deriving information regarding the state of health, for example, because it can be considered whether increased sweating or increased pulse is due to body movement or not. If the unit worn on the body has also been used outside the vehicle, it can thus further be determined how much movement the user had in the time past, whether and how intensely the user has exercised, and the like, which can also be considered in the deriving of information regarding the state of health. Finally, certain movements produce artifacts in the determination of other physiological parameters, which may be recognized as such due to an accompanying monitoring of the movement.

Additionally or alternatively, the physiological parameters received represent the temperature or a heat flux.

It is possible to support the function of the diagnostic module through a variety of other physiological parameters, some of which are explained in detail below. The inventor has recognized, however, that especially the combination of types of information related to heartbeat, heart rate, and/or heart rate variability and electrodermal activity are particularly suitable for the purposes of the invention, preferably in combination with information relating to movement and/or acceleration.

In an advantageous further embodiment, the system comprises at least one of said units worn on the body. In doing so, the unit to be worn on the body may be formed, in particular, by a wristband or a piece of clothing, for example a shirt or a bra, which is equipped with the appropriate sensors. The unit worn on the body preferably comprises one or more of the following sensors: A sensor for determining the heart rate and/or the heartbeat, wherein said sensor is preferably formed by an optical sensor, in particular a photoplethysmography sensor, a sensor for measuring the electrical conductivity of the skin, especially the electrodermal activity, an acceleration sensor, a sensor for measuring the temperature or heat flux, and additionally/alternatively, in the case of a piece of clothing, one or more sensors for detecting an electrocardiogram, the monitoring of respiration, blood pressure, and/or muscle tone.

The unit worn on the body may comprise one or more of the following components or functionalities:
A device for encrypting data transmitted to the receiver of the control unit; In this manner, it can be ensured that the health data is not being accessed by third parties.
A memory for storing physiological parameters of at least the last six hours, preferably at least the last 12 hours, and most preferably at least the last 24 hours; In this manner, physiological parameters may also be obtained outside the vehicle, particularly around the clock, but are collected in the vehicle by the control unit and taken into account by the diagnostic module to derive information regarding the state of health.
A GPS receiver for determining the location of the unit; The GPS receiver can help accurately assess the level of physical activity of the user, for example as the walking or jogging distance traveled and speed in this case. In addition, the GPS receiver can also help in locating the user, regardless of whether those inside or outside the vehicle should have an accident.
A vibration sensor, which can generate a vibration signal perceptible to the wearer; With such a vibration sensor, the user can be alerted to certain states of health or well-being, such as drowsiness. Using this vibration sensor, which is preferably individually adjustable by the user, the user can be reliably alerted when conditions or events occur that place the ability to drive into question.
A means for measuring the blood pressure, a means for electroencephalography, and/or a pulse oximeter for measuring arterial oxygen saturation;

In an advantageous embodiment, the diagnostic module is set up, at least partially based on the physiological parameters received, to derive information regarding one or more of the following states of health or well-being: high stress level, fatigue, exhaustion, drowsiness, loss of consciousness, and/or arrhythmias, wherein the stress level is at least partially determined based on a measured heart rate variability.

Preferably, the control unit is set up to adapt one or more of the vehicle functions to the state of health or well-being in response to the information derived:
A driver assistance system, in particular to maintain greater distances with respect to other vehicles, to reduce the current speed or a possible top speed, or to activate currently inactive assistance functions, such as a lane departure warning system;
An adjustable driving mode or chassis adjustment, in particular a change from a sports to a comfort mode;
A blockage or diversion of incoming calls, text messages, or emails;
A navigation system for finding a calmer route, preferably after consulting with the occupant;
A steering wheel vibration device or other optical or haptic warning devices;
A climate-control system or ventilation system, electric window lever, and/or a sunroof;
A reduction of displays to the necessary functions;
A stereo system, particularly with respect to the volume or selection of music;
An internal illumination, particularly changing the color and/or brightness;

Preferably, the control unit is set up to suggest one or more of the following measures for improving the state of health or well-being of the occupant by means of an output unit of the vehicle:

Suggestion to use autopilot; reminder to take medication; suggestion, preferably via language specification, to take a break, especially in conjunction with subsequent navigation to a parking lot, a rest area, or a cafe; suggestion for fluid or food intake; determination of a calmer route, and suggestion to choose said route.

In an advantageous further embodiment of the invention, the control unit is set up to carry out interactively one of the following measures, which aim at improving the state of health and/or well-being: guided breathing exercises via said output unit, or guided methods of energetic psychotherapy via said output unit, wherein the measures are preferably carried out in an autopilot mode of the vehicle. An example of the methods of energetic psychotherapy is acupressure tapping. However, it is understood that other variants of energetic psychotherapy can be used.

The degree of improvement of the state of health is preferably displayed to the occupant, in particular at least partially based on a measured heart rate variability and/or EDA, during the breathing exercise or the guided acupressure. In this manner, so-called biofeedback is provided to the occupant. With constant stress, the heart rate is also subject to physiological variability, reflecting, among others things, the interaction between the sympathetic and parasympathetic nervous systems. The autonomic nervous system leads, with its sympathetic portion, to a reduced heart rate variability (HRV) by means of the noradrenaline release and, with its parasympathetic or vagal portion, to an increase in HRV by means of the acetylcholine release. The HRV analysis makes it possible to assess this interaction between the sympathetic and parasympathetic nervous systems with different demands.

The system according to this embodiment enables systematic biofeedback, which exploits the close correlation between breathing and heart rate modulation. Such biofeedback methods are known from medicine, for example, from the psychosomatic treatment of stress, depressions, and anxiety. Targeted cardiorespiratory biofeedback enables the reduction of nervousness and tension and allows for concentration and focus at the decisive moment. Since biofeedback can be used easily and free of distraction, it may be used in the car during the drive and not only by passengers but also by the driver, especially during piloted driving.

In an advantageous embodiment, the control unit comprises a memory, wherein the medical data of the occupant are stored. The data may preferably represent one or more of the following types of information here: age, sex, body weight, nicotine consumption, global health, information regarding pre-existing conditions, in particular hypertension, cardiac arrhythmias, heart failure, angina pectoris, previous history of myocardial infarction, and/or mental illnesses, diabetes, information concerning current medication, normal values of physiological parameters or combinations of parameters.

These medical data may be considered by the diagnostic module when deriving the information from the physiological parameters received regarding the state of health in order to increase the reliability of the diagnosis.

Preferably, the control unit is suitable for creating and/or updating the medical data in the memory, in one or more of the following ways: based on the physiological parameters received by the unit worn on the body, in particular based on physiological parameters which were obtained on different days, weeks, or months, by means of an interactive medical history carried out by the control unit or based on external medical data. The external medical data can be, for example, data that are provided by a physician or a telemedicine device.

Preferably, the control unit for exchanging medical data is configured to communicate with at least one of the following devices: a server or a cloud to store personal medical data, a mobile device, on which a program, in particular an app, is installed and is set up for processing medical data, and/or a telemedicine device or a physician's office.

This communication preferably takes place automatically, i.e. without a specific input by the user being needed, who if need be authorizes this communication, but does not necessarily initiate it. Furthermore, this communication is preferably encrypted to prevent access to these medical data by third parties.

Through this means of communication, personal medical data, either on a server or a cloud, are constantly supplemented and updated, either on a portable device or in the database of a physician's office or a telemedicine device, with information, which is represented by the measured physiological parameters or derived therefrom. It is irrelevant whether these physiological parameters were determined in the vehicle or outside the vehicle, which is easily possible using the unit worn on the body. In any case, the vehicle-mounted control or at least the control assigned to the vehicle is used here as a gateway for transmitting these physiological parameters or the information derived therefrom. In this manner, the regular use of the vehicle simultaneously allows regular monitoring of the state of health of the user of the system, who otherwise may not spend the time or have the discipline to determine regularly the physiological parameters and/or to transmit them to a physician or a telemedicine device.

Put another way, this communication allows an updating of the medical data in the memory of the control unit, which, in turn, increases the reliability of the deriving of information regarding the state of health by the diagnostic module.

Although the system according to the invention is based on the use of physiological parameters, which were determined with at least one unit worn on the body, the control unit may continue to be communicatively connected to one or more vehicle-mounted sensors, which enable information or supplementary information related to the state of health, the state of well-being, or illnesses to be derived. This may include, for example, sensors on the steering wheel to measure the body fat content and water content, sensors in the seat to determine the (proportional) body weight, and/or a camera to monitor the eyes.

### BRIEF DESCRIPTION OF THE FIGURES

FIG. 1 shows a schematic representation of a system for monitoring the state of health and/or of well-being of a vehicle occupant, according to an embodiment of the invention;

FIG. 2 is a schematic representation of a unit worn on the body with sensors for recording physiological parameters;

FIG. 3 shows a detailed view of a control unit of the system from FIG. 1;

FIG. 4 shows a flowchart of the operation of the system from FIG. 1; and

FIG. 5 shows a schematic representation of a graphical display of a vital sign and of instructions on breathing exercises;

### DESCRIPTION OF THE PREFERRED EMBODIMENTS

To provide a better understanding of the present invention, it will now be illustrated by some following examples.

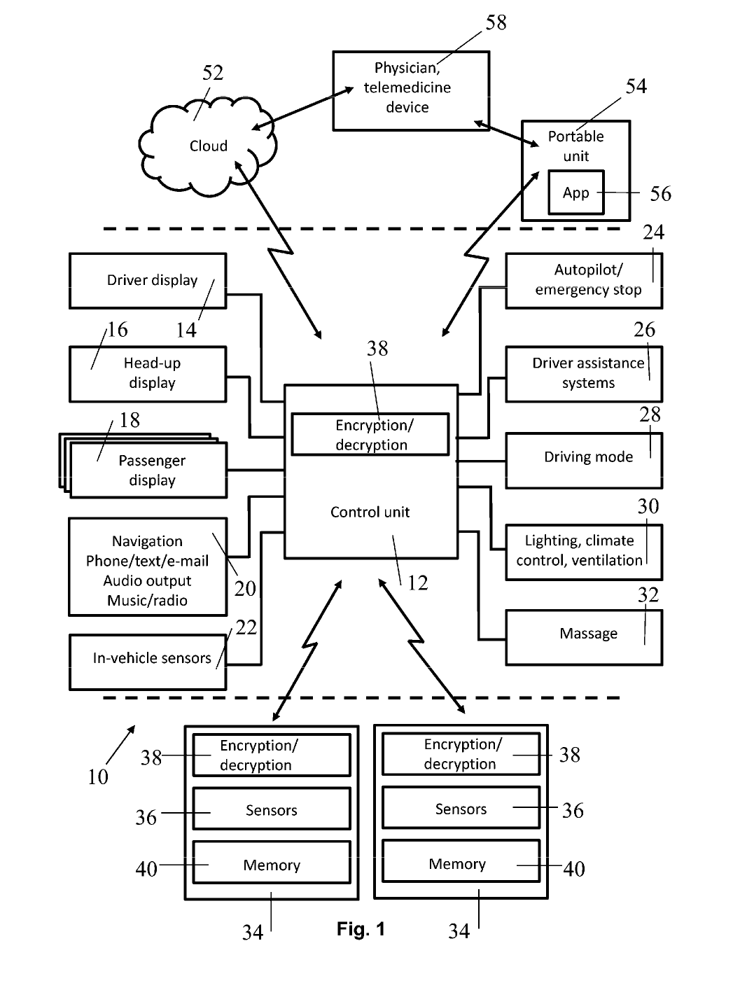

FIG. 1 shows a schematic block diagram of a system 10 for monitoring the state of health and/or of well-being of a vehicle occupant, according to an embodiment of the invention. The vehicle occupant may be either the driver or a passenger. In the following description, reference is made to a passenger car as an example of a vehicle, but the invention is not limited to this; it can be applied to any vehicles, including aircraft.

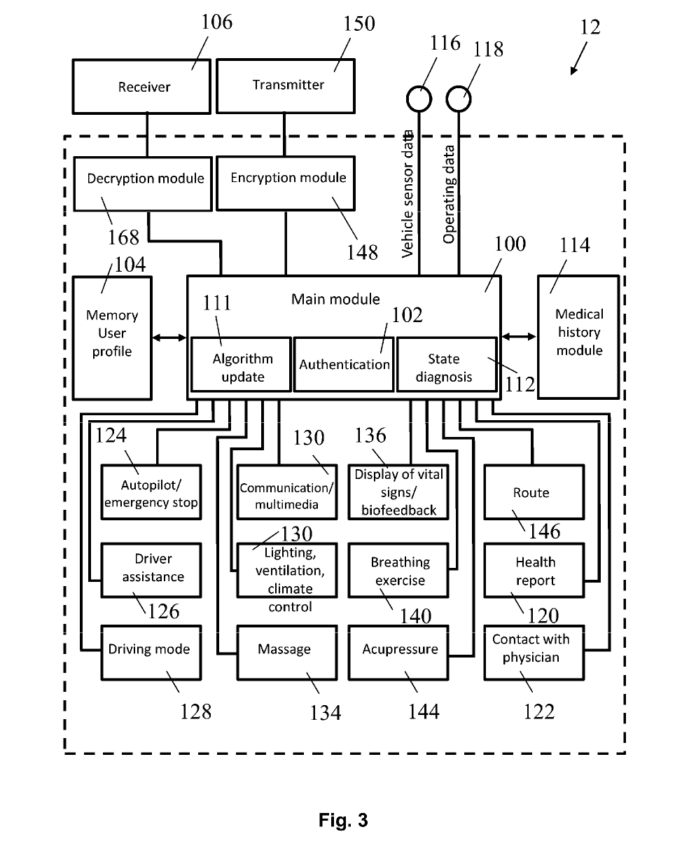

The system 10 comprises one vehicle-mounted control unit 12 assigned to the vehicle, in the specific embodiment. A block diagram showing the control unit in more detail is shown in FIG. 3.

The two horizontal dashed lines in FIG. 1 represent the boundaries of the vehicle. As shown in FIG. 1, the control unit 12 is connected to a plurality of vehicle components via data lines in order to actuate them, and to obtain signals from them if necessary. The components of the vehicle that can be actuated by the control unit 12 include a driver's display 14, a head-up display 16, a plurality of passenger displays 18 for passengers both in the front seat and in the rear, and a so-called communication/multimedia device 20, which combines the functions of a navigation device, a phone, including SMS functionality, an e-mail transmitting and receiving device, an audio output, and music/radio. Instead of an integrated communication/multimedia device, structurally separate individual components may also be provided.

Furthermore, in-vehicle sensors 22 are provided, which allow information or supplementary information to be derived regarding the state of health, the state of well-being, or regarding illnesses of the occupant. Included in these in-vehicle sensors 22 in the preferred embodiment are sensors on the steering wheel for measuring the body fat content and water content of the driver, sensors in the seat for determining body weight, a camera for monitoring the eyes to detect fatigue and/or nodding off of the occupant, and sensors on the seatbelt, which can help, for example, in determining loss of consciousness. All of these in-vehicle sensors are represented in the diagram in FIG. 1 by block 22.

Furthermore, an autopilot device 24 is presented in FIG. 1, which is set up for both autonomous driving and for autonomous implementation of an emergency stop. The autopilot device 24 may also be actuated by the control unit 12, particularly when the control unit 12 determines if there is driver incapacity or impending driver incapacity, for example indication of loss of consciousness, heart attack, stroke, circulatory collapse, or epilepsy.

As can be further seen in FIG. 1, the control unit 12 is connected to driver assistance systems, which are generally represented by block 26.

Furthermore, the vehicle includes a device for adjusting the driving mode 28, a climate-control and ventilation system and interior lighting, which are collectively represented by block 30, as well as a massage device 32.

Furthermore, two units 34 worn on the body are shown schematically in FIG. 1. The units 34 worn on the body contain sensors 36 as essential components designed to determine the physiological parameters of the vehicle occupant. The system can simultaneously include multiple units 34, which are worn by one or more occupants.

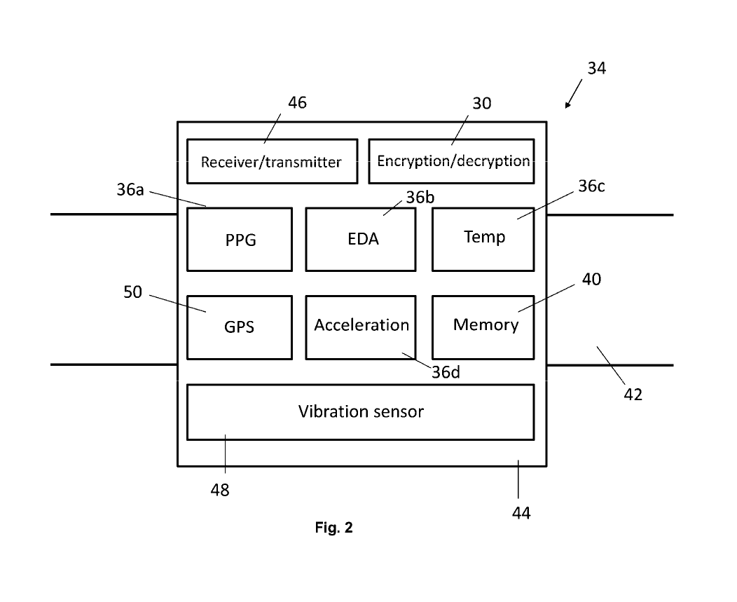

The unit 34 worn on the body may be, for example, a wristband. An example of such a wristband with the corresponding sensors 36 and other components is shown in FIG. 2 and will be described in greater detail below.

The unit worn on the body may be a piece of clothing, in which sensors for detecting physiological parameters are provided, in particular a shirt or bra. As a rule, a single unit worn on the body per user, especially a wristband, is sufficient for the purposes of the invention, depending on requirements, but the system's performance can be increased by using multiple units of this type worn on the body. This is especially recommended for high-risk patients, such as people who have already suffered a heart attack or a stroke or who are suffering from severe diabetes. Currently in Germany, the driver's licenses of about 120,000 of such high-risk patients are suspended temporarily or permanently per year. When the state of health is monitored with the system of the invention, a portion of these patients could continue to drive without unduly endangering themselves and other road users, especially when combined with the integration of the autopilot function 24 into the system.

The units 34 worn on the body also comprise devices 38 for encrypting and decrypting data. In a similar manner, the control unit 12 also contains a device 38 for encrypting and decrypting data. The devices 34 can wirelessly, for example via Bluetooth, communicate with the control unit 12, which is indicated by the radio symbols in FIG. 1. Finally, the units 34 comprise memory 40 for storing physiological parameters. In the preferred embodiment, the memory 40 can store physiological data over a long period of several days, so that the physiological parameters can be recorded for several days around the clock. The stored data can then be transmitted wirelessly to the control unit 12 when the user is using the vehicle.

FIG. 2 schematically shows a wristband as an exemplary embodiment of a sensor unit 34 worn on the body. The device 34 includes a band 42, by means of which the unit 34 can be attached to the wrist, and a housing 44, which contains the aforementioned sensors 36, the encryption device 38, and the memory 40.

In addition, the unit 34 worn on the body includes a receiver/transmitter unit 46 for wireless communication with the control unit 12, a vibration sensor 48, and a GPS receiver 50. The unit 34 may further contain a processor (not shown), which can process the physiological parameters in the unit 34 itself.

The vibration sensor 48 in this case comprises a transducer (not shown), which generates the vibrations of the housing 44, which can be discerned by the wearer of the wristband 34. In this way, the wearer of the wristband can be made aware or warned of certain states, for example when the user is about to fall asleep, or when there are signs of impending driver incapacity, so that the user is still able to stop the vehicle safely.

The sensors 36 comprise a sensor 36a for determining physiological parameters, which represent the heartbeat, the heart rate, or heart rate variability of the user. In the embodiment shown, it is a photoplethysmography sensor, for generating a photoplethysmogram (PPG). Using the sensor 36a, heartbeats can be detected and consequently the heart rate and/or the heart rate and/or the time interval between two successive heartbeats. In particular the HRV can be derived from this, which either occurs in the unit 34 itself or in the control unit 12 based on the time information of the heartbeats.

The wristband 34 in FIG. 2 also includes a sensor 36b for measuring the electrical conductivity of the skin, specifically the electrodermal activity. Furthermore, the wristband 34 includes a sensor 36c for measuring the temperature of the skin or a heat flux, and an acceleration sensor 36d.

The measurement of the acceleration is used, among other things, to prevent artifacts that can be caused by movement of the user, especially when measuring the HRV. If an acceleration or movement is detected by the acceleration sensor 36d, the other physiological parameters simultaneously measured can be optionally ignored or cut out so as not to corrupt the measurement results due to movement-induced artifacts.

However, the acceleration sensor 36d can also provide information about the movement of the user outside the vehicle, thus if necessary determine, together with the information of the GPS 50, how far and how fast the user has walked or jogged in a past time period and the like. This information is useful both in terms of global health monitoring as well as with regard to the correct interpretation of current and stored physiological parameters.

In another reference to FIG. 1, it is shown that the control unit 12 is further configured for the exchange of medical data to communicate with a cloud 52 and a portable device 54, which may be, for example, a smartphone or a tablet. In the exemplary embodiment shown, an app is installed on the portable device 54, which is represented by the reference number 56 and is set up for processing medical data. Furthermore, the system 10 is set up to communicate with a physician's office or a telemedicine device, which are schematically represented by the block 58 in FIG. 1. As can be seen in FIG. 1, personal medical data can be exchanged between the physician/telemedicine device 58 and the cloud 52 on one side and the portable device 54 on the other side. Although not explicitly shown in FIG. 1, it is also possible for the control unit 12 to communicate directly with the physician/telemedicine device 58 in one variant.

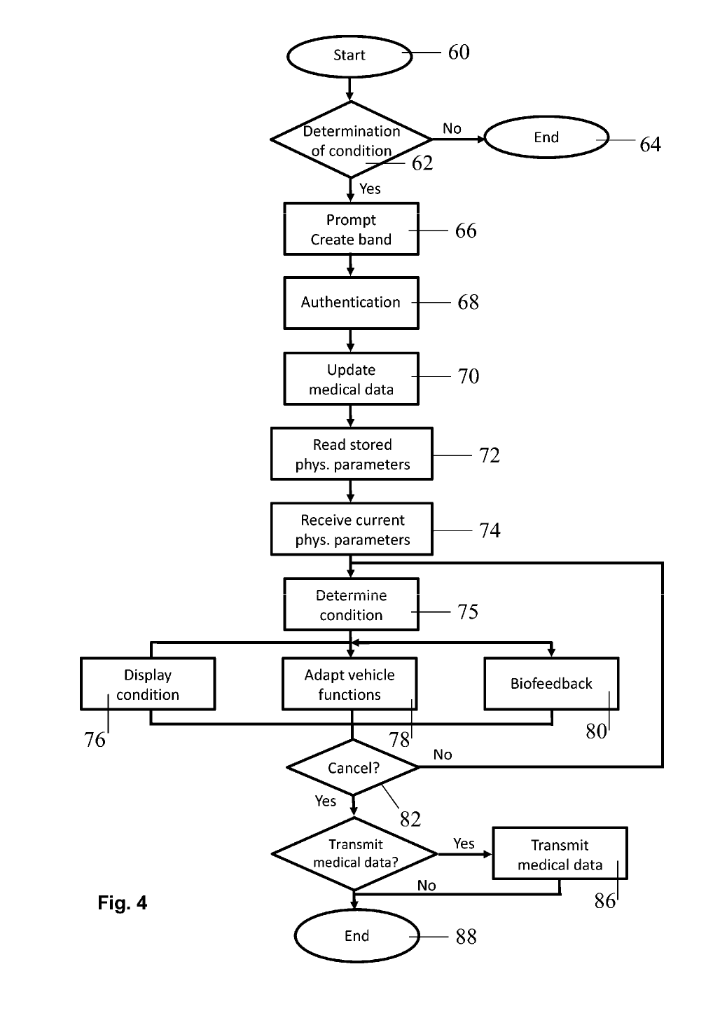

The function of the system 10 will next be described in more detail with reference to FIGS. 3 and 4. FIG. 3 shows a block diagram of the control unit 12 here, in which the modules and functions of the control unit 12 are shown in more detail. FIG. 4 shows a flowchart, which illustrates the sequence of a method according to an embodiment of the invention.

The method starts in step 60, for example, when the vehicle engine is started.

In step 62, the user is asked whether he/she wants to determine his/her current state of health/well-being based on the measurement of certain physiological parameters. The question can be generated either via speech output with the assistance of the audio output device 20 (see FIG. 1) of the communication/multimedia device or via optical indication on the driver's display 14 or, for a passenger, on the passenger's display 18. The driver can respond to this question either by voice input or by input on a touchscreen or the like. If the driver rejects the determination of the state of health, the method proceeds to step 64 and ends there. Otherwise, the method proceeds to step 66, in which the control unit 12 of the system 10 prompts the user to apply the wristband 34 shown schematically in FIG. 2. In the following step 68, the user is authenticated. To authenticate, an RFID chip or an NFC chip can be used, which is assigned to the user or the unit 34 worn on the body. Alternatively, the user may also authenticate, for example, by entering a code or the like. In an expanded embodiment, the user may also be identified by his/her individual, biometric parameters, which were determined using the sensors worn on the body.

As shown in FIG. 3, a main module 100 of the control unit 12 comprises an authentication module 102. In the present disclosure, the term “module” generally refers to functional units in a broad sense, regardless of whether these are implemented through hardware units or software modules.

In the following step 70, medical data are updated. In the exemplary embodiment shown, the medical data are stored in a memory unit 104, which contains a user profile. The medical data here represent information regarding age, sex, body weight, nicotine consumption, global health, information regarding pre-existing conditions, particularly hypertension, cardiac arrhythmias, heart failure, angina pectoris, previous history of myocardial infarctions, mental illnesses, and/or diabetes, information regarding current medication, and normal values of certain physiological parameters or combinations of parameters. The updating of the medical data may, on one hand, be done from external medical data that are sourced from the cloud 52, from the portable device 54, or from the physician and/or the telemedicine device 58. These data are received by means of a receiver 106 shown in FIG. 3 and decrypted using a decryption module 108, which is part of the encryption/decryption device 38 generally shown in FIG. 1. Based on the updated data, an algorithm update module 110 executes an update of algorithms, which uses a state diagnostic module 112 also contained in the main module 100, in order to derive information regarding the state of health, the state of well-being, or illnesses based on the physiological parameters received. The state diagnostics module 112 thus uses a “learning” algorithm. If the user has been prescribed a beta blocker, for example, since the last use of the system 10, the heart rate and/or heart rate variability should be assessed differently than it would without this information. In this respect, the algorithm update is important in order to always draw the right conclusions from the physiological parameters.

Furthermore, in step 70, a medical history module 114 can be used, which takes an interactive voice-controlled medical history with the user, to update the medical records. This is particularly advantageous during the initial use of the system.

In the following step 72, physiological parameters are read, which are stored in the memory 40 of the unit 34 worn on the body and have been determined in a certain previous time period (for example, in the last 24 hours) or since the last use of the system. These data provide something of a “history,” for example, as to whether the user has been exposed to extensive physical or emotional stress in the past 24 hours, or cardiac abnormalities, or too much/too little physical movement, etc. This information can also be stored in the memory 104 for the user profile and be considered during the updating of the algorithms, which are used by the state diagnostic module 112.

In the subsequent step 74, the reception of the current physiological parameters starts. This primarily involves parameters that are determined by the sensors 36 in the unit 34 worn on the body, in particular physiological parameters representing the heartbeat, the heart rate, and/or the HRV of the vehicle occupant and parameters representing the electrodermal activity. Depending on the type of units 34 worn on the body that are used, other physiological parameters can also be received, wherein multiple such units 34 can be used simultaneously by the same user, as has already been shown in FIG. 1. In addition to the physiological parameters that were already mentioned in connection with the description from FIG. 2, a number of other physiological parameters can be considered. In particular, complete electrocardiograms can be obtained using sensors that are integrated into pieces of clothing, which is of enormous practical importance, breathing and blood pressure can be very closely monitored, and/or a bioimpedance analysis can be performed. Depending on the medical history, other physiological parameters can be determined; for example, brain waves (EEG), muscle tone, blood sugar, and possibly even laboratory values can be determined using mobile measuring instrumentation and methods (e.g. DrySpot, rHEALTH technologies, etc.).

The physiological parameters transmitted by the unit 34 worn on the body are also received by means of the receiver 106 and decrypted using the decryption module 108. This should be considered a simple representation; however, usually separate receivers and separate decryption modules can be and are used in practice.

In addition to the physiological parameters, which are calculated using the unit 34 worn on the body, the main module 100 still receives vehicle sensor data and vehicle operational data. Appropriate inputs, 116 and 118, are provided for this purpose. The vehicle sensor data are data that have been determined with vehicle-mounted sensors and that enable information or supplemental information to be derived regarding the state of health, the state of well-being, or illnesses. This may be, for example, sensors on the steering wheel to measure the body fat content and water content, or sensors in the seat to determine the (proportional) body weight. The determined body weight can then be compared to the body weight data from the user profile memory 104, and, in this way, weight fluctuations can be found, which may be an indication of water retention in the body. Another example of a vehicle-mounted sensor is a camera for observing the eyes to detect drowsiness or nodding off in the driver.

The operating data may, for example, relate to speed, RPM, braking behavior, steering movements, and the like. Depending on these operational data, the main module 100 can analyze the driver's driving style and consider it in further operation, for example, detect a very aggressive driving style or find that the current driving style requires the driver's full attention and refraining from all activities that require interaction. Another example is the detection of driver inactivity indicating drowsiness.

In the following step 75, information regarding the state of health, the state of well-being, or illness is determined by the state diagnostics module 112 based on the physiological parameters received.

In particular, at step 75, information relating to a stress level, fatigue, exhaustion, drowsiness, loss of consciousness, and/or cardiac arrhythmias is derived. In the preferred embodiment, the stress level in this case is at least partially determined based on a measured heart rate variability.

Based on the current physiological parameters received, the system 10 executes three procedures in a time-parallel manner in the method shown in FIG. 4 below, namely the display of the current state in step 76, the adaptation of vehicle functions in step 78, and biofeedback applications in step 80.

In sub-process 76, the user is shown information regarding his/her condition, either visually on the driver's display 14 or by voice output via the audio output function of the multimedia device 20 (see FIG. 1). The output can be a simple, intuitive display, for example a “stress level”, which can be represented using a bar graph or a color code (for example, red for a high stress level, green for low). In the embodiment shown, however, the control unit comprises a health report module 120, which can give the user a detailed voice-controlled report about his/her current state of health.

The health report can also contain advice to consult with a physician and possibly obtain an appointment and provide the relevant medical data in advance. Additionally or alternatively, audio/video contact can be established with the physician or therapist using the communication/multimedia device 20 in the vehicle. For such contact with the physician, the control unit 12 includes a physician contact module 122.

Sub-process 78 is executed in parallel with sub-process 76 and relates to the adaptation of vehicle functions to the state of health or well-being of the user determined in step 75. If the diagnosis of the state diagnosis module 112 determines signs of driver incapacity, particularly signs of loss of consciousness, heart attack, stroke, circulatory collapse, or epilepsy, the autopilot/emergency device 24 shown in FIG. 1 is actuated by an autopilot/emergency stop module 124 of the control unit 12 in order to execute an emergency stop.

The autopilot/emergency stop module 124 of the control unit 12 instructs the autopilot device 24 to take over with an autonomous driving mode. At the same time, the autopilot/emergency stop module 124 gives the driver a warning, via the driver's display 14, the head-up display 16, or an audio output, with the offer to reject the intervention of the vehicle if the state diagnostics module 112 has misinterpreted the signs of driver incapacity and the driver is actually still fit and able to drive. If the driver does not react, however, an autonomous emergency stop is initiated, in which the autopilot device 24 steers the vehicle autonomously to the roadside. At the same time, an emergency call is sent out, for example on the multimedia device 20. The hazard warning lights are simultaneously activated to warn other vehicles. In an advantageous embodiment, a “medical emergency” OLED display is activated in the rear window. The autopilot/emergency stop module 124 of the control unit 12 can furthermore arrange for the personal medical data from the memory 104 to be transmitted to the emergency physician in the form of an electronic medical record and for information related to physiological parameters, particularly those parameters that have been evaluated as being an indicator of driver incapacity by the state diagnostics module 112 of the control unit 12, to be transferred. In this manner, the emergency physician can obtain essential information regarding the type of incident on the way to the emergency and, at the emergency site, act quickly and effectively and/or request additional assistance in advance.

A further adaptation of vehicle functions in sub-process 78 relates to the adaptation of driver assistance systems 20 (see FIG. 1), which is controlled by a driver assistance module 126 of the control unit 12.

For example, if the state diagnostic module 112 determines that the driver shows signs of stress, exhaustion, or fatigue, the driver assistance module 126 controls selected driver assistance systems 26 in order to adapt them to the state of health or well-being. In particular, the driver assistance system 26 is adjusted to maintain larger distances with respect to other vehicles and to slow down the current speed or possible maximum speed. In addition, the driver assistance module 126 may activate assistance functions currently inactive for the driver that can relieve the driver: for example, a lane departure warning system.

Also as part of sub-process 78, a driving mode module 128 of the control unit 12 checks if the current driving mode should be adapted to the driver's state of health or well-being. If the state diagnostic module 112 recognizes, for example, signs of fatigue, exhaustion, or stress, the driving mode module 128 may suggest switching to a different driving mode, for example, switching from a sports mode to a comfort mode, as is possible with many current vehicles. In particular, the chassis setting can be adjusted from a stiffer to a more comfortable suspension to accommodate the current state of the driver.

Also as part of sub-process 78, a communication/multimedia module 130 checks whether the current settings of the communication and multimedia device 20 of FIG. 1 should be adapted to the state determined by the state diagnostics module 112. In the event of extensive exertion or stress on the driver, incoming calls, text messages, or e-mails, for example, can be blocked or diverted to relieve the driver. Furthermore, the module 130 can actuate the navigation component of the multimedia device 20 to determine a calmer route to the destination, i.e. a route with less traffic, which may take a little longer, but promises a less strenuous drive, and offer this to the driver. Without having to point this out individually each time, it is understood that the modules that are intended to control the adaptation of vehicle functions first suggest the adaptation to the user and only actually carry out the action after confirmation by the user.

Furthermore, the communication module 130 may actuate the stereo component of the communication multimedia device 20 according to the state of well-being, for example, reduce the volume or play certain preset playlists with music that the driver considers to be calming. To further reduce stress on the driver, the control unit 12 can restrict the number of displayed indicators to a few necessary functions to further relieve the driver.

Furthermore, a module 132 is provided, which adapts the lighting, climate-control, and ventilation function 30 in FIG. 1 to conform to the determined state of health or well-being of the driver, so, for example, if there are signs of fatigue, it increases the supply of oxygen, for example through stronger ventilation, opening windows or the sunroof, or supplying stored oxygen. The color of the interior lighting can also be adjusted according to the current state of well-being, for example to colors that are perceived by the driver as being stimulating or soothing depending on the situation. It is also possible to offer the driver light therapy, in particular to offer a so-called “light shower”. This is based on the finding that many people need light to be awake and feel fit and to suppress the release of melatonin. To stop the formation of melatonin, a light intensity of a few thousand lux, for example 10,000 lux, is required.

Another adaptation of vehicle functions to the determined state of health or well-being state relates to the offering and optionally performing of a massage by means of a massage device 23 arranged in the seats under the control of a massage module 134.

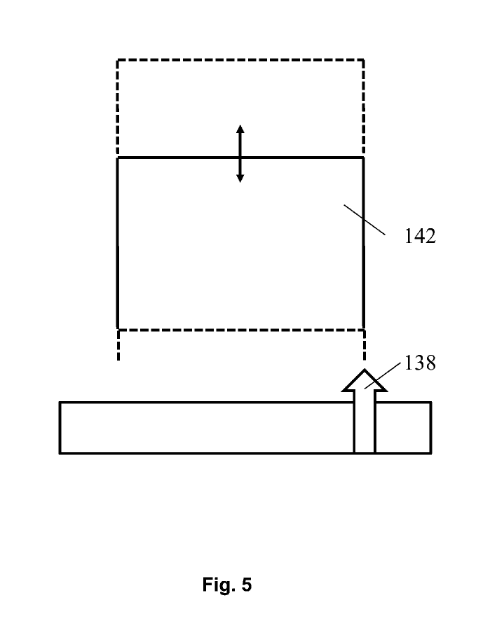

Furthermore, a biofeedback sub-process is executed in parallel to sub-processes 76 and 78, which is represented by the reference number 80. The control unit 12 comprises a module 136, which shows the display of a vital sign on the driver's display 14 or the head-up display 16 (for the driver) or on a passenger's display 18 (for a passenger). This vital sign is used to give the user clear information about his/her current state of health. FIG. 5 schematically shows a corresponding display in the form of a pointer 138. In the preferred embodiment, the vital sign is based, at least partially, on the measured heart rate variability. In the diagram shown, a inclination of the pointer 138 to the right means a high stress level, while a position further to the left means a lower stress level.

The concept of “biofeedback” is based on making a person aware of changes in condition variables of biological processes that are not accessible to direct sensory perception. At the same time, it is possible for the user to influence these variables, for example through corresponding breathing depth and frequency, and thereby improve his/her well-being. In addition to displaying the vital sign using the pointer 138, thus a breathing exercise module 140 is provided, which displays inhalation and exhalation cycles to the user that help to improve the vital sign. In the representation of FIG. 5, a bar 142 is displayed together with the pointer 138 for this. A slow rise of the bar 142 to the upper dashed position indicates the inhalation action to the user. A lowering of the bar to the lowest position, which is likewise dashed, indicates the exhalation action. During the breathing guided by the breathing exercise module 140, the vital sign is measured continuously, and the user can see how it develops using the pointer 138. Actually, it is possible to improve the heart rate variability significantly through precisely guided breathing in a comparatively short time, whereby the well-being of the user is increased.

To show a more meaningful vital sign with the pointer 138, it is, in turn, advantageous that medical data are stored in the memory 104, for example, averages of past heart rate variability measurements or the like. It is also possible for the user to view long-term trends related to the vital sign. As mentioned at the outset, it is of particular importance here that the measurement of the vital sign always take place in the same environment, meaning that it depends on comparatively few environmental influences, whereby a comparability of measurements is increased at different times.

In addition, the graphical display of the pointer 138 and the breathing bar 142 is simply and easily detectable to the extent that they hardly distract the driver while driving, so that the breathing exercise can be carried out during the drive itself. Preferably, however, the biofeedback applications, especially the breathing exercises, are performed in phases of autonomous driving, whereby the time saved can be put to beneficial use.

In the embodiment shown, an acupressure module 144 is also provided, which guides the user interactively to perform acupressure tapping, also accompanied by the display of the vital sign with assistance of the pointer 138.

In addition to biofeedback applications, the control unit 12 may suggest additional measures to improve the state of health or well-being of the occupant. For example, the control unit 12 can remind the user of medication according to the medical information stored in the memory 104. In addition, the control unit 12 can suggest that the user use the autopilot when an increased stress level, fatigue, or exhaustion are detected, or the control may suggest to the driver, via voice input, to take a break, especially in conjunction with subsequent navigation to a parking lot, a rest area, or a cafe, or search automatically for a calmer route using a route module 146, and, if there is a practical calmer route, suggest this to the driver.

In step 82, there is a check as to whether the process should be aborted. This is the case if the user rejects all suggestions in all sub-procedures 76, 78, and 80. As long as this is not the case, the process returns to step 75 and goes through the processes described again.

If it is decided in step 82 that the process should be aborted, there is a query in subsequent step 84 as to whether newly obtained medical data should be transmitted. These are data based on the last measured physiological parameters and characterize the current state of the health of the user. These data include special events such as the occurrence of cardiac arrhythmias or the like.

If the user agrees to the transfer of the medical data, medical data are transmitted in step 86. To this end, the data are encrypted in the encryption module 148 and sent to the cloud 52 (see FIG. 1), to a portable device 54, or a physician or telemedicine device 58 via the transmitter 150. Although only one encryption module 148 and one transmitter 150 are shown in FIG. 3, it is understood that multiple encryption modules and transmitters may be provided, which may transmit the data to different receivers via different channels.

As is apparent from the present detailed description, the system and method provide multiple advantages according to various embodiments of the invention. By combining multiple sensors and physiological parameters, artifacts can be very well detected and corrected. By combining different methods in the analysis of measurement data and through the use of learning algorithms, an improvement in the accuracy and individual adaptation to the respective users and their respective condition or their respective level of physical activity is achieved.

Using the units worn on the body, not only the accuracy of the measurement can be increased, but also physiological parameters when the user is not in the vehicle can be determined, in particular around the clock.

By implementing the system of the invention, the vehicle virtually becomes the medical partner and provides the interface between users and physicians, medical call centers, telemedicine, emergency physicians, etc. Furthermore, it is also possible for passengers to use the system in the preferred embodiment.

A consultation with the physician or therapist is possible via a secure audio/video contact using the multimedia system in the car, whereby the time in the car, especially in autonomous driving mode, can be optimally utilized. The vehicle is also suitable as an ideal space for regular determination of physiological parameters, which can be used to generate long-term analyses and health reports. The reason for this is that similar comparable conditions always prevail in the vehicle to the extent that most users use the vehicle regularly and for a comparatively long period of time and that the vehicle conveys a private atmosphere in which the user can deal with his/her health in an uninhibited and unobserved manner. In this respect, the vehicle offers a space and an interface for health monitoring for those who would otherwise not have the opportunity to address this because of their lifestyle.

Although many aspects of the present invention have been described specifically in connection with applications in passenger vehicles, the invention is not limited to these.

In particular, the invention also has important applications in the aerospace industry related to monitoring the state of health of pilots. In this case, the pilot would also wear the sensors directly on the body, preferably in the form of a wristband, but additionally or alternatively in the form of a piece of clothing, which is equipped with sensors and is worn directly on the body. In this manner, the system can provide very early warnings of psychological or health-critical situations, for example, high stress levels, heart attack, stroke, loss of consciousness, arrhythmias, epilepsy, hypoglycemia, fatigue, drowsiness, depression relapses, etc. The combination of a sensor for determining the heart rate, in particular a photoplethysmography sensor and a sensor for measuring the electrical conductivity of the skin, in particular the electrodermal activity, preferably further combined with an acceleration sensor, is in turn particularly suitable, especially for use by pilots. The measurement of physiological parameters essentially takes place in the same manner as described above. It is also advantageous for the pilot if the control unit informs him/her about his/her state of health, but this is not absolutely necessary in this specific application.

All variants and applications described above can be transferred to use in an aircraft to the extent they do not specifically reference passenger car components.

Especially for pilots, the advantage of sensors, which are provided in at least one unit worn on the body, is of special significance, particularly for the comparison of physiological parameters outside of the aircraft and during the flight. In this manner, sudden, big changes in the state of health of the pilot can be easily identified.

While the monitoring of the state of health during use in a passenger car primarily aims to inform users themselves, the monitoring of the state of health of the pilots obviously is of paramount importance to the airline and its passengers who entrust their lives to the pilot. For this reason, there can be a provision specifically for flight applications that the physiological parameters or the information derived therefrom be sent regularly to third parties for monitoring purposes, for example, persons or entities within the airline can monitor the pilot's fitness to fly. In this manner, for example, increased stress levels, fatigue, etc. can be addressed quickly. If the values are critical, the pilot could possibly get issued a temporary flying ban while still on the ground. During the flight, it can be ensured that the pilot is replaced early enough by a colleague even if the pilot does not request this.

In sudden medical emergencies, such as heart attack, stroke, or loss of consciousness, the autopilot immediately takes control and issues an emergency message to the crew and the respective appropriate air traffic control. This is another example of the aforementioned general concept to adapt “vehicle features” within the system of the invention, in this case, the autopilot, to the determined state of health or the detected event. In particular, a provision may be for the autopilot to take over control automatically until the nearest airport.

The system can also detect increased fatigue or nodding off of both pilots early on, which could be caused, for example, by toxic gases in the cockpit air. The autopilot in this case also takes over the controls and issues an emergency signal. Additionally or alternatively, the cockpit air can also be replaced with clean air at an early stage.

Furthermore, as described previously when there is a high level of stress, appropriate measures for stress reduction are offered to the pilot, for example biofeedback methods or guided methods of energetic psychotherapy, for example acupressure tapping.

As emphasized above, the system of the invention is not limited to use by the vehicle operators, for example, motorists or pilots, but it is also aimed at the passengers. The guided breathing exercises or biofeedback or guided methods of energetic psychotherapy are of great importance especially for passengers, particularly those with a fear of flying. For this purpose, a plane seat can be placed, for example in an airport lounge, on which the user can be instructed in the use of the system in place at that time on the aircraft, under the guidance of a member of the ground crew.

### REFERENCE NUMBERS

10 System
12 Control unit
14 Driver display
16 Head-up display
18 Passenger display
20 Multimedia device
22 Sensors
24 Autopilot device
26 Driver assistance system
28 Driving mode device
30 Climate-control and ventilation system, interior lighting
32 Massage device
34 Unit worn on the body
36 Sensors
38 Units for encryption and decryption
40 Memory
42 Band
44 Housing
46 Receiver/transmitter unit
48 Vibration sensor
50 GPS receiver
52 Cloud
54 Portable unit
56 App
58 Telemedicine device
60-88 Steps in the method acc. to an embodiment of the invention
100 Main module
102 Authentication module
104 User profile memory
106 Receiver
108 Authentication module
110 Update module
112 State diagnostics module
114 Medical history module
118 Transmitter
116/118 Inputs
120 Health report module
122 Physician contact module
124 Autopilot/emergency stop module
126 Driver assistance module
128 Driving mode module
130 Communication/multimedia module
132 Module for air, climate control, and ventilation
134 Massage module
136 Vital signs module
138 Pointer
140 Breathing exercise module
142 Bar
144 Acupressure module
146 Routes module
148 Encryption module
150 Transmitter

1-13. (canceled)

14. A system for monitoring the state of health and/or of well-being of a vehicle occupant, comprising:
a control unit that is assigned to and mounted to a vehicle, the control unit comprising:
a receiver configured to wirelessly receive physiological parameters of the vehicle occupant from a wearable unit comprising one or more sensors configured to determine the physiological parameters, a heart rate, a heartbeat, or a heart rate variability of the vehicle occupant,
a diagnostic module—configured to derive information regarding the state of health, the state of well-being, or an illness of the vehicle occupant based on the physiological parameters received by the receiver,
wherein the control unit is configured to provide the derived information to the vehicle occupant at the output unit and to initiate at least one of:
adapting at least one vehicle function based on the derived information,
suggesting, at the output unit, measures to improve the state of health, the state of well-being, or the illnesses of the vehicle occupant, or
interactively carrying out measures, at the output unit, to improve the state of health, the state of well-being, or the illnesses of the vehicle occupant,
wherein the measures are carried out while the vehicle is in an autopilot mode and comprise a breathing exercise guided by the output unit or an acupressure exercise guided by the output unit, and
wherein the control unit is further configured to display a degree of improvement of the state of health, wherein the degree of improvement is based, at least partially, on a heart rate variability of the vehicle occupant during the breathing exercise or the guided acupressure.

15. The system according to claim 14, wherein the diagnostic module is further configured to determine signs of driver incapacity, based on the physiological parameters received by the receiver, and in response thereto, instruct an autopilot device of the vehicle to perform an emergency stop and initiate an emergency call, wherein the signs of driver incapacity include a loss of consciousness, a heart attack, a stroke, a circulatory collapse, or epilepsy.

16. The system according to claim 14, wherein the physiological parameters further include an electrodermal activity, a movement, a heart-rate acceleration, a temperature flux, or a heat flux.

17. The system according to claim 14, wherein the wearable unit is a wristband, a bra, or a shirt, and the wearable unit comprises at least one of:
a sensor configured to determine the heart rate, wherein the sensor configured to determine the heart rate is an optical sensor or a photoplethysmography sensor;
a sensor configured to measure an electrical conductivity of a portion of skin of a wearer of the wearable unit or an electrodermal activity;
an acceleration sensor;
a sensor configured to measure a temperature or a heat flux; or
one or more sensors, embedded in an article of clothing, configured to determine an electrocardiogram, monitor breathing, measure blood pressure, or monitor muscle tone.

18. The system according to claim 17, wherein the wearable unit comprises one or more of the following:
a device configured to encrypt data transmitted to the receiver of the control unit;
a memory unit configured to store the physiological parameters;
a GPS receiver configured to determine a location of the wearable unit;
a vibration unit configured to generate a vibration signal perceptible to a wearer of the wearable unit;
a blood pressure sensor configured to measure a blood pressure;
a sensor configured to perform an electroencephalography; or
a pulse oximeter.

19. The system according to claim 14, wherein the information derived by the diagnostic module includes information regarding at least one of: a stress level, fatigue, exhaustion, drowsiness, a loss of consciousness, or an arrhythmia, wherein the stress level is at least partially determined based on a measured heart rate variability and an electrodermal activity (EDA).

20. The system according to claim 14, wherein the control unit is configured to adapt, based on the derived information at least one of:
a driver assistance system to maintain greater distances with respect to other vehicles, to reduce the current speed or a top speed, or to activate inactive assistance functions;
an adjustable driving mode or chassis adjustment from a sports to comfort mode;
a blockage or diversion of incoming calls, text messages, or emails;
a navigation system for finding a calmer route, after consulting with the occupant;
a steering wheel vibration device or other optical or haptic warning devices;
a climate-control or ventilation system;
an electric window lever or a sunroof;
a display indications system to provide reduced indications;
a stereo system, configured to adjust the volume or a selection of music; or
an internal lighting system, to change a color or a brightness of the internal lighting system.

21. The system according to claim 14, wherein the measures to improve the state of health, the state of well-being, or the illness of the vehicle occupant, comprise at least one of:
a suggestion to use an autopilot;
a reminder to take medication;
a suggestion to take a break in conjunction with subsequent navigation to a parking lot, a rest area, or a cafe;
a suggestion to ingest at least one of a fluid or a food; or
a determination of a calmer route and a suggestion to choose the calmer route.

22. The system according to claim 14, wherein the control unit is configured to perform automatic communication and/or encrypted communication of medical information with at least one of the following devices:
a server configured to store personal medical data;
a cloud configured to store personal medical data;
a mobile device comprising an application configured to process medical data;
a telemedicine device; or
a physician's office.

23. The system according to claim 14, wherein the control unit is connected to one or more vehicle-mounted sensors that derive information related to the state of health, the state of well-being, or the illnesses of the vehicle occupant, the one or more vehicle-mounted sensors comprising at least one of:
sensors on a steering wheel configured to measure a body fat content and a water content;
a seat sensor configured to determine a proportional body weight of the vehicle occupant; or
a camera configured to monitor eyes.

24. A method for monitoring the state of health and/or the well-being of a vehicle occupant, comprising:
determining physiological parameters of the vehicle occupant using one or more sensors arranged on a wearable unit worn by the vehicle occupant;
transmitting, via a wireless connection, the physiological parameters to a control unit assigned to and mounted to a vehicle;
deriving information regarding at least one of a state of health, a state of well-being, or an illnesses of the vehicle occupant based at least partially on the physiological parameters;
informing the vehicle occupant, using at least one output unit, about the state of health, the state of well-being, or the illness of the vehicle occupant;
initiating at least one of the following: adapting at least one vehicle function based on the state of health, the state of well-being, or the illness of the vehicle occupant, suggesting, at the output unit, measures to improve the state of health, the state of well-being, or the illness of the vehicle occupant, or
interactively carrying out measures to improve the state of health, the state of well-being, or the illness of the vehicle occupant,
wherein the measures are carried out while the vehicle is in an autopilot mode and comprise a breathing exercise guided by the output unit or an acupressure exercise guided by the output unit; and
displaying a degree of improvement in the state of health, wherein the degree of improvement is based, at least partially, on a measured heart rate variability of the vehicle occupant during the breathing exercise or the guided acupressure.

25. The method according to claim 24, wherein the physiological parameters comprise at least one of a heart rate variability or an electrodermal activity.

26. The method according to claim 24, wherein transmitting the physiological parameters to the control unit further comprises encrypting the physiological parameters and automatically transmitting the encrypted one or more physiological parameters to at least one of the following:
a server configured to store personal medical data;
a cloud configured to store personal medical data;
a mobile device comprising an application configured to process medical data;
a telemedicine device; or
a physician's office.
